(19) United States
(12) Patent Application Publication
(10) Pub. No.: US 2003/0153612 A1
(43) Pub. Date: Aug. 14, 2003

(54) Method of treatment or prophylaxis of restless legs syndrome with ropinirole compound

(75) Inventor: Kapil Dev Sethi, Augusta, GA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 10/361,252

(22) Filed: Feb. 10, 2003

Related U.S. Application Data
(63) Continuation of application No. 10/105,118, filed on Mar. 22, 2002, now abandoned.
Application No. 10/105,118 is a continuation of application No. 09/879,462, filed on Jun. 12, 2001, now abandoned.
Application No. 09/879,462 is a continuation of application No. 09/342,760, filed on Jun. 29, 1999, now abandoned.
(60) Provisional application No. 60/091,088, filed on Jun. 29, 1998.

(51) Int. Cl. A61K 31/405

(52) U.S. Cl. 514/418

(57) ABSTRACT

The present invention provides a method of treating Restless Legs Syndrome in human or animal patient, which method comprises administering to said patient an effective amount of ropinirole or a pharmaceutically acceptable salt or solvate thereof. Preferably, a dose of ropinirole or a pharmaceutically acceptable salt or solvate thereof is administered to the patient 1 to 3 hours before the patient goes to bed. A typical dose comprises 0.1 mg -5 mg of ropinirole. The invention also provides a pharmaceutical composition for use in the treatment of Restless Legs Syndrome which comprises an effective amount of ropinirole or a pharmaceutically acceptable salt or solvate thereof and a pharmaceutically acceptable carrier.

### FIELD OF THE INVENTION

The present invention relates to a method of treatment or prophylaxis, and has particular reference to a method of treating or preventing Restless Legs Syndrome (RLS).

### BACKGROUND TO THE INVENTION

Restless Legs Syndrome (also known as Ekbom syndrome) is a common condition that is regularly associated with stereotypical jerks of the lower limbs during sleep. RLS is often desired by sufferers as an unpleasant creeping sensation, likened to “crawling ants” or “writhing worms” in the muscles and bones which usually occurs in the evenings. The sensations usually occur in the calf, sometimes in the thighs and feet, and rarely in the arms. Once the sensations begin, there is an overwhelming need to move to release the feelings and general discomfort. This need to move occurs on average every 20 to 40 seconds, and the movements last for about 1 to 5 seconds. RLS affects about 5% of the population at some time. Men and women are equally affected, but it is more common in the elderly. RLS usually follows a chronic cause, but it may occur in pregnancy and then remit. Most cases of RLS are reported to be idiopathic, but relationships with other diseases are known. It has been associated with iron deficiency anaemia and uraemia (where it may precede uraemic neuropathy). Up to 30% of patients with rheumatoid arthritis may suffer from RLS, and it has been reported in 30% of patients with fibromyalgia. It may also be associated with poliomyelitis, ovitamenosis, diabetes, smoking, Parkinson's Disease and lengthy exposure to cold.

The origins of the symptoms of RLS are poorly understood. There are no detectable changes in the muscles or nerves. EEG studies have demonstrated repetitive arousals in RLS with Kα complexes before the onset of movement.

Historically, the treatment of RLS has been empirical. Narcotics have been used, but obviously have limitations. Well controlled studies in this indication are rare and, prior to 1987, only carbamazepine and clonazepam have been shown to be superior to placebos. In the mid 1980s, treatment with open label L-dopa was reported with positive results. These results have since been replicated in several placebo controlled studies using doses of L-dopa of 100 mg to 200 mg. L-dopa has been shown to be effective in 70% of patients, and the effect has been maintained for 2 years.

There is therefore a need to provide alternative treatments for Restless Legs Syndrome, particular for patients where treatment with L-dopa is ineffective or only partially effective.

There is also a general need to provide an improved treatment for Restless Legs Syndrome.

### OBJECTS OF THE INVENTION

An object of the present invention is to provide a new method of treating, or preventing Restless Legs Syndrome.

Another object of the present invention is to provide an improved method of treating or preventing Restless Legs Syndrome.

### SUMMARY OF THE INVENTION

According to one aspect of the present invention there is provided a method of treatment or prophylaxis of Restless Legs Syndrome in an human or animal patient, which method comprises administering to said patient an effective amount of ropinirole or a pharmaceutically acceptable salt or solvate thereof.

In another aspect of the present invention there is provided the use of ropinirole or a pharmaceutically acceptable salt or solvate thereof in the manufacture of a medicament for the treatment or prophylaxis of Restless Legs Syndrome in human and animals.

RLS patients treated with ropinirole have reported a dramatic improvement in their condition, almost at once.

Ropinirole is 4-[2-(di-n-propylamino)ethyl]-1,3-dihydro-2H-indolin-2-one hydrochloride. This compound is disclosed in U.S. Pat. No. 4,452,808 and U.S. Pat. No. 4,588,740, where it is disclosed to have antihypertensive and anti-anginal properties. U.S. Pat. No. 4,824,860 and U.S. Pat. No. 4,912,126 disclose that ropinirole is a potent CNS active non-ergot dopamine receptor antagonist. The hydrochloride salt of ropinirole is approved for human use in therapy to treat Parkinson's Disease.

Ropinirole used in the present invention is suitably in the form of the freebase or a pharmaceutically acceptable salt thereof. A preferred pharmaceutically acceptable salt of ropinirole is the crystalline hydrochloride. Suitable procedures for preparing ropinirole hydrochloride include those mentioned in U.S. Pat. No. 4,997,954, and preferably those mentioned in U.S. Pat. No. 5,336,781.

A medicament for use in the treatment or prophylaxis of RLS may be prepared by a mixture of ropinirole or a pharmaceutically acceptable salt or solvate thereof with an appropriate carrier, which may contain a diluent, binder, filler, disintegrant, flavouring agent, colouring agent, lubricant or preservative in conventional manner.

Preferably, the medicament is in unit dosage form and in a form adapted for use in the medical or veterinarial fields. For example, such preparations may be in a pack form accompanied by written or printed instructions for use as a treatment for RLS.

The suitable dosage for ropinirole or a pharmaceutically acceptable salt or solvate depends on the severity of the RLS in any given patient and on the condition of that patient. It will also depend, inter alia, upon the relation of potency to absorbability and the frequency and route of administration.

Typically the patient in need of treatment or prophylaxis in accordance with this invention will not be suffering from Parkinson's disease and/or will not be receiving treatment for Parkinson's disease using ropinirole.

### DETAILED DESCRIPTION OF THE INVENTION

Ropinirole or a pharmaceutically acceptable salt or solvate thereof may be formulated for administration by any route, and examples are oral, sub-lingual, rectal, topical, parenteral, intravenous or intramuscular administration. Preparations may, if desired, be designed to give slow release of the ropinirole or a pharmaceutically acceptable salt or solvate thereof.

The medicaments may, for example, be in the form of tablets, capsules, sachets, vials, powders, granules, lozenges, reconstitutable powders, or liquid preparations, for example solutions or suspensions, or suppositories.

The medicaments, for example those suitable for oral administration, may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycerine; tabletting lubricants, for example magnesium stearate; disintegrants, for example starch, polyvinylpyrrolidone, sodium starch glycollate or microcrystalline cellulose; or pharmaceutically acceptable setting agents such as sodium lauryl sulphate.

Solid medicaments may be obtained by conventional methods of blending, filling, tabletting or the like. Repeated blending operations may be used to distribute ropinirole or a salt or solvate thereof throughout those medicaments employing large quantities of fillers. When the medicament is in the form of a tablet, powder, or lozenge, any carrier suitable for formulating solid pharmaceutical compositions may be used, examples being magnesium stearate, starch, glucose, lactose, sucrose, rice flour and chalk. Tablets may be coated according to methods well known in normal pharmaceutical practice, in particular with an enteric coating. The medicament may also be in the form of an ingestible capsule, for example of gelatin containing ropinirole or a salt thereof if desired with a carrier or other excipients.

Medicaments for oral administration as liquids may be in the form of, for example, emulsions, syrups, or elixirs, or may be presented as a dry product for reconstitution with water or other suitable vehicle before use. Such liquid medicaments may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; aqueous or non-aqueous vehicles, which include edible oils, for example almond oil, fractionated coconut oil, oily esters, for example esters of glycerine, or propylene glycol, or ethyl alcohol, glycerine, water or normal saline; preservatives, for example methyl or propyl p-hydroxybenzoate or sorbic acid; and if desired conventional flavouring or colouring agents.

Ropinirole or a pharmaceutically acceptable salt or solvate thereof may also be administered by a non-oral route. In accordance with routine pharmaceutical procedure, the medicaments may be formulated, for example for rectal administration as a suppository. They may also be formulated for presentation in an injectable form in an aqueous or non-aqueous solution, suspension or emulsion in a pharmaceutically acceptable liquid, e.g. sterile pyrogen-free water or a parenterally acceptable oil or a mixture of liquids. The liquid may contain bacteriostatic agents, anti-oxidants or other preservatives, buffers or solutes to render the solution isotonic with the blood, thickening agents, suspending agents or other pharmaceutically acceptable additives. Such forms will be presented in unit dose form such as ampoules or disposable injection devices or in multi-dose forms such as a bottle from which the appropriate dose may be withdrawn or a solid form or concentrate which can be used to prepare an injectable formulation.

As mentioned hereinbefore, the effective dosage of the ropinirole or pharmaceutically acceptable salt or solvate thereof depends on the severity of RLS to be treated, the condition of the patient and on the frequency and route of administration.

Preferably, the composition is administered in the form of one or more dosage units. A dosage unit for oral administration may comprise 0.1 mg to 50 mg of ropinirole; preferably 0.25 mg-5 mg. For parenteral administration, a dosage unit may comprise 0.1 mg-15 mg of ropinirole.

The daily dosage of ropinirole for an adult patient may be between 0.1 mg and 100 mg orally, preferably between 0.25 mg and 50 mg, and more preferably between 0.25 mg and 15 mg; or an intravenous, subcutaneaus or intramuscular dosage of between 0.1 mg and 50 mg, preferably between 0.25 mg and 15 mg, of ropinirole. The compound may be administered 1 to 4 times per day as required, typically 1, 2 or 3 times per day. Usually the compound will be administered for a period of continuous therapy. The daily dosage of ropinirole may be increased throughout the period of therapy if a patient develops a level of tolerance to the drug.

For patients showing nighttime symptoms, an evening dose of ropinirole is preferably administered shortly before the patient goes to bed, typically 1-3 hours before going to bed. In some cases, this evening dose may comprise 0.25- 0.5 mg of ropinirole, at least initially. In patients with solely nighttime symptoms, this may be a sufficient daily regimen to obtain relief from the symptoms. In patients with symptoms throughout the day, 1-3 doses of ropinirole may be administered at convenient or suitable intervals throughout the day. The total daily dosage may be 0.25-12 mg, or 0.25-6 mg. For example, where a second dose is required in addition to the evening dose, it may be administered earlier in the day, preferably in late afternoon. It is preferred that ropinirole is be taken either during or after a meal. A typical regimen for the treatment of RLS comprises the administration of two or three 0.25-2 mg doses of ropinirole per day. The doses may be administered at intervals of 3 to 7 hours, typically 5 hours. For example, one regimen comprehends the administration of 0.5 mg of ropinirole three times per day at 12:00 am, 5:00 pm and 10:00 pm. Another example regimen comprises the administration of 2 mg of ropinirole 3 times per day.

The present invention may be practised using a controlled release or delayed release formulation containing ropinirole or a pharmaceutically acceptable salt thereof. By “controlled release” is meant any formulation technique wherein release of the active substance from the dosage from is modified to occur at a slower rate than that from an immediate release product, such as a conventional swallow tablet or capsule.

By “delayed release” is meant any formulation technique wherein release of the active substance from the dosage form is modified to occur at a later time than that from a conventional immediate release product. The subsequent release of active substance from a delayed release formulation may also be controlled as defined above.

Examples of controlled release formulations which are suitable for incorporating ropinirole are described in:

Sustained Release Medications, Chemical Technology Review No. 177. Ed. J. C. Johnson. Noyes Data Corporation 1980, and

Controlled Drug Delivery, Fundamentals and Applications, 2nd Edition. Eds. J. R. Robinson, V. H. L. Lee. Marcel Dekker Inc. New York 1987.

Examples of delayed release formulations which are suitable for incorporating ropinirole are described in:

Remington's Pharmaceutical Sciences 16th Edition, Mack Publishing Company 1980, Ed. A. Osol.

The present invention further provides a pharmaceutical composition for use in the treatment of RLS which comprises an effective amount of ropinirole or a pharmaceutically acceptable salt or solvate thereof and a pharmaceutically acceptable carrier. Such compositions may be prepared in the manner as hereinbefore described.

1. A method of treatment or prophylaxis of Restless Legs Syndrome in an human or animal patient, wherein said method comprises administering to said patient an effective amount of ropinirole or a pharmaceutically acceptable salt or solvate thereof.

2. A method as claimed in claim 1, wherein ropinirole is administered in the form of ropinirole hydrochloride.

3. A method as claimed in claim 1, wherein ropinirole or a pharmaceutically acceptable salt or solvate thereof is administered to said patient for a period of continuous therapy.

4. A method as claimed in claim 1, wherein said ropinirole or pharmaceutically acceptable salt or solvate thereof is administered in the form of one or more dosage units.

5. A method as claimed in claim 4, wherein a doasge unit for oral administration comprises 0.1 mg-50 mg of ropinirole or a pharmaceutically acceptable salt or solvate thereof.

6. A method as claimed in claim 4 wherein a dosage unit for oral administration comprises 0.25 mg-5 mg of ropinirole or a pharmaceutically acceptable salt or solvate thereof.

7. A method as claimed in claim 4 wherein a doasge unit for parental administration comprises 0.1 mg-15 mg of ropinirole or a pharmaceutically acceptable salt or solvate thereof.

8. A method as claimed in claim 1 wherein a patient is given a daily oral dosage of 0.1 mg-100 mg of ropinirole or a pharmaceutically acceptable salt or solvate thereof.

9. A method as claimed in claim 1 wherein a patient is given a daily oral dosage of 0.25 mg-15 mg of ropinirole or a pharmaceutically acceptable salt or solvate thereof..

10. A method as claimed in claim 1 wherein ropinirole or a pharmaceutically acceptable salt or solvate thereof is administered 1 to 4 times per day.

11. A method as claimed in claim 1, wherein an evening dose of ropinirole or a pharmaceutically acceptable salt or solvate thereof is administered to a patient 1 to 3 hours before said patient goes to bed for the treatment or prophylaxis of nighttime symptoms.

12. A method as claimed in claim 11, wherein a second afternoon dose is administered to the patient 3 to 7 hours before the evening dose.

13. A method as claimed in claim 1, wherein ropinirole or a pharmaceutically acceptable salt or solvate thereof is administered to said patient at 3 to 7 hour intervals throughout the day.

14. Use of ropinirole or a pharmaceutically acceptable salt or solvate thereof in the manufacture of a medicament for use in the treatment or prophylaxis of Restless Legs Syndrome in a human or animal patient.

15. A medicament for use in the treatment or prophylaxis of Restless Legs Syndrome, which medicament is prepared by mixing ropinirole or a pharmaceutically acceptable salt or solvate thereof with an appropriate carrier.

16. A medicament as claimed in claim 15, wherein said medicament is provided in pack form accompanied by written or printed instructions for use as a treatment or prophylaxis for Restless Legs Syndrome.

17. A pharmaceutical composition for use in the treatment or prophylaxis of Restless Legs Syndrome in a human or animal patient, which composition comprises an effective amount of ropinirole or a pharmaceutically acceptable salt or solvate thereof and a pharmaceutically acceptable carrier.

18. The method of claim 3, wherein the daily dosage amount of ropinirole is increased throughout the period of treatment.

19. The method of claim 11, wherein said evening dose comprises 0.25-0.5 mg ropinirole.

20. A method as claimed in claim 1, wherein a patient is administered 1 to 3 doses of ropinirole throughout the day for the treatment or prophylaxis of daytime symptoms.

21. The method of claim 20, wherein the total daily dosage of ropinirole is 0.25-12 mg.

22. The method of claim 20, wherein the total daily dosage of ropinirole is 0.25-6 mg.

23. The method of claim 20, wherein each dose of ropinirole comprises 0.25-2 mg.

24. The method of claim 20, wherein the doses are administered at 5 hour intervals.

25. The method of claim 1, wherein the patient is not suffering from Parkinson's disease.
